(19) United States
(12) Patent Application Publication
(10) Pub. No.: US 2025/0177762 A1
(43) Pub. Date: Jun. 5, 2025

(54) WIRELESS CHARGER FOR IMPLANTABLE MEDICAL DEVICE

(71) Applicant: Greatbatch Ltd., Clarence, NY (US)

(72) Inventors: Luis Daniel Villamil, Montevideo (UY); Camila Duarte, Montevideo (UY); Gabriel Amilcar Barbat, Montevideo (UY); Pablo Morales, Montevideo (UY); Andres Duarte, Montevideo (UY); Rocio Belen Curiel, Montevideo (UY); Lucia L. Sirio, Montevideo (UY)

(21) Appl. No.: 18/964,756

(22) Filed: Dec. 2, 2024

Related U.S. Application Data
(60) Provisional application No. 63/604,921, filed on Dec. 1, 2023.

(51) Int. Cl. A61N 1/378; H02J 50/00; H02J 50/10

(52) U.S. Cl. CPC A61N 1/3787; H02J 50/005; H02J 50/10

(57) ABSTRACT

In various examples, a wireless charger for an implantable medical device includes a housing including an interior. The housing includes a bottom side configured to face the patient proximate the location of the implantable medical device. A rechargeable battery is disposed within the interior of the housing. The battery is configured to store electricity. A coil is disposed proximate the bottom side of the housing and electrically coupled to the battery. The coil is configured to inductively couple with the implantable medical device with the charger placed against the patient proximate the location of the implantable medical device in order to inductively charge the implantable medical device using electricity from the battery.

### CROSS REFERENCE TO RELATED APPLICATIONS

This application claims the benefit of priority to U.S. Provisional Application Ser. No. 63/604,921, filed on Dec. 1, 2023, entitled “WIRELESS CHARGER,” which is incorporated by reference herein in its entirety.

### BACKGROUND

Traditional chargers for implantable medical devices typically require cables and usually include two separate devices: a coil and a charger unit attached to the coil with a cable. These devices can limit mobility and be cumbersome to carry around. On the other hand, chargers in which the coil and charger are combined in a single enclosure are typically cumbersome and require a harness to maintain the charger in place with respect to the patient, which can limit patient mobility while wearing such a harness.

### OVERVIEW

This overview is intended to provide an overview of subject matter of the present patent document. It is not intended to provide an exclusive or exhaustive explanation of the invention. The detailed description is included to provide further information about the present patent document.

The present inventors have recognized, among other things, that the present inventive subject matter can be used to charge an implantable medical device implanted within a patient while minimally impacting comfort and/or mobility of the patient. In various examples, the present inventive subject matter is advantageous in that it provides a rechargeable charger that allows for cordless charging of the implantable medical device when the charger is sufficiently charged. Moreover, in some examples, the present inventive subject matter provides for a single-component compact charger that can be easily attached to the patient in the location of the implantable medical device and concealed underneath clothing of the patient. In this way, in various examples, the present inventive subject matter is advantageous in that it provides a charger that is relatively easy to attach to the patient, relatively easy to control and monitor, relatively comfortable to wear, and minimally impactful to the mobility of the patient while wearing the charger. To better illustrate the devices described herein, a non-limiting list of examples is provided here:

Example 1 can include subject matter that can include a wireless charger for an implantable medical device. The implantable medical device is implantable at a location within a patient. The charger includes a housing including an interior. The housing includes a bottom side configured to face the patient proximate the location of the implantable medical device. A rechargeable battery is disposed within the interior of the housing. The battery is configured to store electricity. A coil is disposed proximate the bottom side of the housing and electrically coupled to the battery. The coil is configured to inductively couple with the implantable medical device with the charger placed against the patient proximate the location of the implantable medical device in order to inductively charge the implantable medical device using electricity from the battery.

In Example 2, the subject matter of Example 1 optionally includes a control module disposed within the interior of the housing. The control module is configured to selectively supply electricity from the battery to the coil in order to inductively charge the implantable medical device.

In Example 3, the subject matter of Example 2 optionally includes a communication module configured to communicate with a mobile device. With the mobile device communicatively coupled to the communication module, the mobile device is configured to communicate with the control module and control the charger.

In Example 4, the subject matter of Example 3 is optionally configured such that the mobile device includes an application configured to allow a user to control the charger.

In Example 5, the subject matter of any one of Examples 1-4 optionally includes an adhesive member attached to the charger. The adhesive member is configured to adhere the charger to the patient during charging of the implantable medical device.

In Example 6, the subject matter of Example 5 is optionally configured such that the adhesive member is removably attachable to the charger.

In Example 7, the subject matter of any one of Examples 1-6 is optionally configured such that the coil is disposed within the interior of the housing proximate the bottom side of the housing.

In Example 8, the subject matter of Example 7 is optionally configured such that the coil is disposed proximate and around a side wall of the housing.

In Example 9, the subject matter of any one of Examples 1-8 is optionally configured such that the coil is disposed along the bottom side of the housing.

In Example 10, the subject matter of any one of Examples 1-9 is optionally configured such that the coil is embedded within the bottom side of the housing.

In Example 11, the subject matter of Example 10 is optionally configured such that the coil is molded within a portion of the bottom side of the housing.

Example 12 can include, or can optionally be combined with any one of Examples 1-11 to include subject matter that can include a wireless charger for an implantable medical device. The implantable medical device is implantable at a location within a patient. The charger includes a housing including an interior. The housing includes a bottom side configured to face the patient proximate the location of the implantable medical device. An adhesive member is attached to the charger. The adhesive member is configured to adhere the charger to the patient during charging of the implantable medical device. A rechargeable battery is disposed within the interior of the housing. The battery is configured to store electricity. A control module is disposed within the interior of the housing. A coil is disposed proximate the bottom side of the housing and electrically coupled to the battery. The coil is configured to inductively couple with the implantable medical device with the charger placed against the patient proximate the location of the implantable medical device. The control module is configured to selectively supply electricity from the battery to the coil in order to inductively charge the implantable medical device.

In Example 13, the subject matter of Example 12 optionally includes a communication module configured to communicate with a mobile device. With the mobile device communicatively coupled to the communication module, the mobile device is configured to communicate with the control module and control the charger.

In Example 14, the subject matter of Example 13 is optionally configured such that the mobile device includes an application configured to allow a user to control the charger.

In Example 15, the subject matter of any one of Examples 12-14 is optionally configured such that the adhesive member is removably attachable to the charger.

In Example 16, the subject matter of any one of Examples 12-15 is optionally configured such that the coil is disposed within the interior of the housing proximate the bottom side of the housing.

In Example 17, the subject matter of Example 16 is optionally configured such that the coil is disposed proximate and around a side wall of the housing.

In Example 18, the subject matter of any one of Examples 12-17 is optionally configured such that the coil is disposed along the bottom side of the housing.

In Example 19, the subject matter of any one of Examples 12-18 is optionally configured such that the coil is embedded within the bottom side of the housing.

Example 20 can include, or can optionally be combined with any one of Examples 1-19 to include subject matter that can include a wireless charger for an implantable medical device. The implantable medical device is disposed at a location within a patient. The charger includes a housing including an interior. The housing includes a bottom side configured to face the patient proximate the location of the implantable medical device. An adhesive member is removably attachable to the charger. The adhesive member is configured to adhere the charger to the patient during charging of the implantable medical device. A rechargeable battery is disposed within the interior of the housing. The battery is configured to store electricity. A control module is disposed within the interior of the housing. A communication module is configured to communicate with a mobile device. With the mobile device communicatively coupled to the communication module, the mobile device is configured to communicate with the control module and control the charger. A coil is disposed proximate the bottom side of the housing and electrically coupled to the battery. The coil is configured to inductively couple with the implantable medical device with the charger placed against the patient proximate the location of the implantable medical device. The control module is configured to selectively supply electricity from the battery to the coil in order to inductively charge the implantable medical device.

### BRIEF DESCRIPTION OF THE DRAWINGS

FIG. 1 is simplified depiction of a charger positioned on a patient to charge an implantable medical device, the charger being in accordance with at least one example of the invention.

FIG. 2 is a perspective view of a charger in accordance with at least one example of the invention.

FIG. 3 is a perspective view of the charger of FIG. 2 with an adhesive member removed from a housing of the charger.

FIG. 4 is a perspective view of the charger of FIG. 3.

FIG. 5 is a front view of a mobile device configured to control a charger in accordance with at least one example of the invention.

FIG. 6 is an exploded perspective view of a charger in accordance with at least one example of the invention.

FIG. 7 is a cross-section of a charger in accordance with at least one example of the invention.

FIG. 8 is a cross-section of a charger in accordance with at least one example of the invention.

FIG. 9 is an exploded perspective view of a bottom side of a housing of a charger in accordance with at least one example of the invention.

FIG. 10 is a perspective view of a coil of the charger of FIG. 9.

### DETAILED DESCRIPTION

The present inventive subject matter relates generally to a charger for a medical device. More specifically, the present inventive subject matter relates to a wireless, mobile charger for charging an implantable medical device. In various examples, the charger is less cumbersome than previous chargers, such that a patient can wear the charger and move about without having a cable plugged into an outlet or having to wrestle with multiple components of the charger or bulky devices attached to the patient.

The present inventive subject matter, in some examples, is directed to a battery powered, wireless, wearable charger that provides a convenient and portable solution for charging an implantable medical device, such as, but not limited to, an implantable pulse generator device. More specifically, in some examples, the charger includes a single component that can be securely attached to a patient, for instance, using an adhesive member, such as, but not limited to, an adhesive patch, an adhesive surface, an adhesive substance, or the like associated with the charger. In this way, in some examples, the charger of the present inventive subject matter allows for hands-free charging while on the move without compromising or significantly impacting patient comfort.

The purpose of the present inventive subject matter, in some examples, is to create a single compact charging device that provides a more comfortable experience for the user, which can be monitored using, for instance, an app on a mobile device. In some examples, the charger includes a coil within the charger to facilitate a smaller device and that can be placed almost anywhere on a body of a patient thanks to an adhesive member configured to adhere the charger to the patient, providing a secure and comfortable charging experience. In some examples, the charger of the present inventive subject matter combines both parts of a typical charger (that is, the coil and the charger) into one singular compact device that can be fully controlled and monitored through an app on a mobile device. Furthermore, in some examples, attachment of the charger to the patient using an adhesive member allows the charger to be adapted to each patient's needs and body in a simple and inexpensive way. In some examples, the present inventive subject matter provides a more flexible, convenient, and user-friendly charging solution that can be easily checked and controlled by an app on a mobile device.

In contrast to traditional charging devices, the charger of the present inventive subject matter, in some examples, reduces, if not eliminates, the need for cables and combines the two components (coil and charger) into one compact device with self-support, offering enhanced mobility and flexibility to the patient when wearing the charger. In some examples, both components of a typical charging device, the charger and the coil header, are merged into one singular device/component: a charger with a coil incorporated inside. An adhesive member, in some examples, can provide a secure attachment to various body surfaces of the patient, depending upon the location of the implantable medical device within the patient, thereby allowing for uninterrupted charging while maintaining patient comfort.

Furthermore, in some examples, the charger can be connected to a mobile device, such as, but not limited to a smart phone or other cellular telephone, a smart tablet, a computer, etc. via an app on the mobile device, with which the patient and/or a care giver can monitor proper alignment of the charger with the implantable medical device, monitor charging status of the implantable medical device and the charger, control monitoring and/or therapy settings and/or parameters of the implantable medical device, among other things.

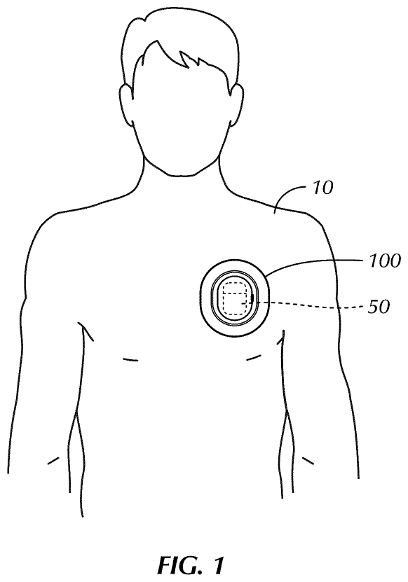

Referring now to the drawings wherein like reference numerals identify similar structural elements or features of the subject invention, there is illustrated in FIG. 1 a wireless charger 100 configured to charge an implantable medical device 50 implanted at a location within a patient 10. The charger 100, in various examples, is configured to attach to the patient 10, for instance, the skin of the patient 10 in alignment with the medical device 50 in order to charge the medical device 50. The implantable medical device 50, in various examples, can include various types of implantable medical devices, such as, but not limited to, a cardiac implantable pulse generator, a pacemaker, an implantable defibrillator, a neurostimulator, a cardiac monitor, a cochlear implant, etc. Although shown implanted within a chest of the patient 10, in various examples, the medical device 50 can be implanted at various locations within the patient 10, including, but not limited to, a chest, a skull, a limb, a back, a pelvic region, etc. of the patient, with the charger 100 being configured to be selectively attached to the patient 10 at the location of the medical device 50 in order to properly charge the medical device 50.

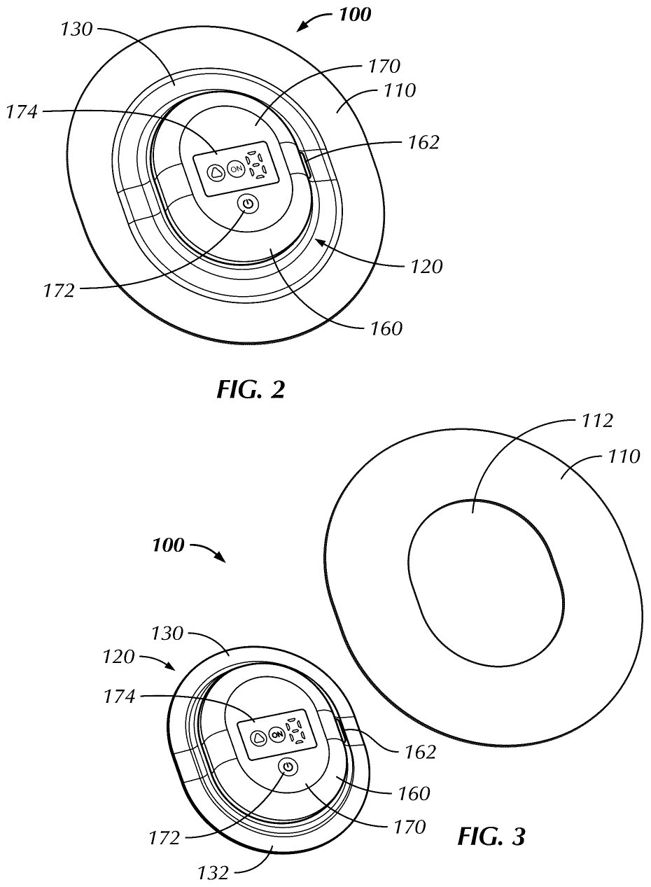

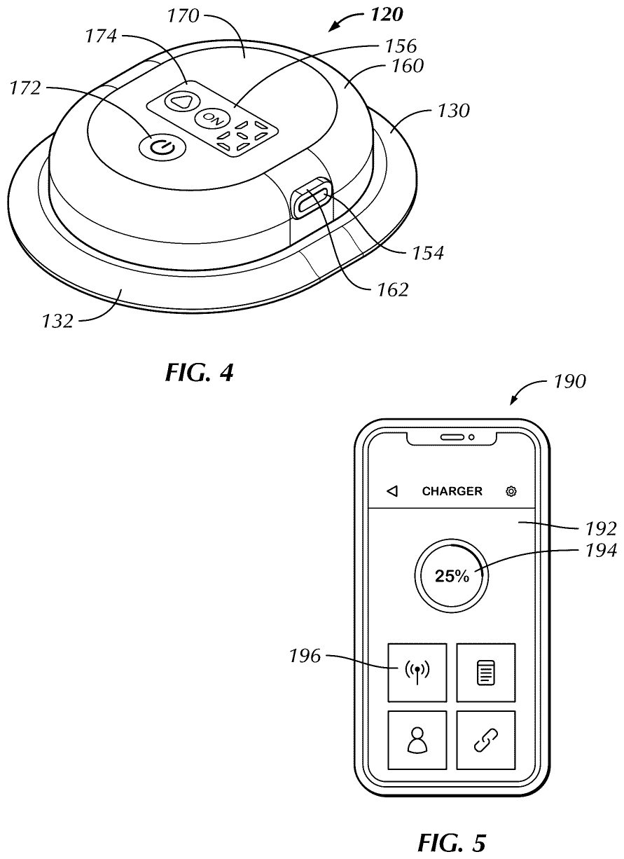

Referring now to FIGS. 1-4, in some examples, the charger 100 is wireless in that there are no wires, cords, or cables needed to attach the charger 100 to power while the charger 100 is attached to the patient 10 in position to charge the medical device 50 implanted within the patient 10. Furthermore, in some examples, the charger 100 is configured to wirelessly couple to the medical device 50 in order to wirelessly charge the medical device 50. In this way, in some examples, the charger 100 is relatively small and compact, allowing for increased mobility of the patient 10 while wearing the charger 100 and/or charging the medical device 50 and reducing, if not eliminating, the necessity of the patient 10 to remain sitting or otherwise disposed in one spot and/or wearing an unwieldy, bulky, and/or heavy charger while charging, as is often the case using a typical charger.

The charger 100, in some examples, includes a housing 120 sized and shaped to fit within components of the charger 100 (as described herein) and facilitate the patient 10 wearing the charger 100 in the location of the medical device 50. The charger 100, in some examples, includes an adhesive member 110 attached to the charger 100. In some examples, the adhesive member 110 includes an adhesive patch or other substrate configured to be attached to the housing 120. In some examples, the adhesive member 110 is configured to adhere the charger 100 to the patient 10 during charging of the medical device 50. In some examples, the adhesive member 110 is removably attachable to the charger 100. In this way, the adhesive member 110 can be replaced periodically to ensure that a functioning adhesive member 110 is used to allow the charger 100 to be affixed to the patient 10. That is, in some examples, the adhesive member 110 is removable and can be replaced with another adhesive member 110 once the patient 10 or caregiver has finished using it. In some examples, the adhesive member 110 is substantially ring-shaped and configured to fit around the housing 120 with the housing 120 being disposed within a hole 112 in the adhesive member 110, such that the adhesive member 110 is attached to and extends around a perimeter of the housing 120 with the housing 120 extending through the hole 112 of the adhesive member 120. In other examples, the adhesive member can include a shape other than substantially ring shaped, including, but not limited to, one or more elongate strips, a cuff, a U-shaped member, or the like, to name a few, depending upon the location of attachment for the charger and/or the shape and/or configuration of the housing 120. In some examples, the adhesive member 110 is adhesively affixed to the housing 120. In some examples, the housing 120 includes a bottom surface 130 configured to contact the skin of the patient. In some examples, the bottom surface 130 includes a lip 132 extending radially outwardly from the housing 120 upon which the adhesive member 110 can be removably adhered.

The adhesive member 110, in some examples, ensures a secure and comfortable fit on various body surfaces as well as body types. In various examples, patients can pick and choose the shape and type of the adhesive patch that better accommodates their needs. Unlike traditional chargers that require devices to be secured with harnesses or bands, the wearable charger 100 offers flexibility in terms of where and how a medical device can be charged. Patients can continue their daily activities while their respective medical devices are being charged without the hassle of holding or securing their chargers with harnesses or bands. In some examples, the adhesive member 110 is removable and can be replaced with another patch when needed to ensure proper securement of the charger 100 to the patient 10. In some examples, the adhesive member 110 is separate from the housing 120 so that the adhesive member 110 can hold the charger 100 in place during a charging session and be replaced by another adhesive member 110 once the charging session is finished or the previous adhesive member 110 otherwise becomes less than optimal for reuse. Compared to typical charger, the charger 100 and adhesive member 110 of the present inventive subject matter, in some examples, are two separate components. The adhesive member 110 is not a carrier with adhesive tabs or part of the charger 100 itself. In some examples, the adhesive member 110 is a fully adhesive patch that is configured to adhere to both the charger 100 and the patient 10. In further examples, the adhesive member 110 can be an off-the-shelf patch that sticks to the body and holds the charger 100 in place.

In some examples, the charger 100 includes a top surface 160 outwardly-facing from the patient 10. The top surface 160, in some examples, forms a compact, ergonomic shape of the charger 100 sized and shaped to fit the components (described herein) of the charger 100 within the charger 100. In some examples, the top surface 160 includes a charging feature 162 formed within the top surface 160 that includes an opening in the housing 120 to allow access to a charging port 154 for selectively plugging in a charging cable when the charger 100 is to be charged. The charging port 154, in various examples, can include various configurations, including, but not limited to, a USB port, a USB-C port, a mini USB port, a micro USB port, a custom port, or the like.

In some examples, the housing 120 further includes a faceplate 170 attached to the top surface 160. The faceplate 170, in some examples, is snapped into engagement with the top surface 160. In some examples, the faceplate 170 includes a power button 172 to allow the patient 10 to power on and power off the charger 100. In some examples, the power button 172 can include a light or other indicator to denote when the charger 100 is powered on. In some examples, the faceplate 170 includes a window 174 or an opening through which to allow the patient to view a display 156 disposed below. In various examples, the display 156 can show various information to the patient 10 or caregiver, including, but not limited to, charge level, charge time, an error indicator, a warning indicator, a temperature indicator, an alignment indicator, or the like, to name a few.

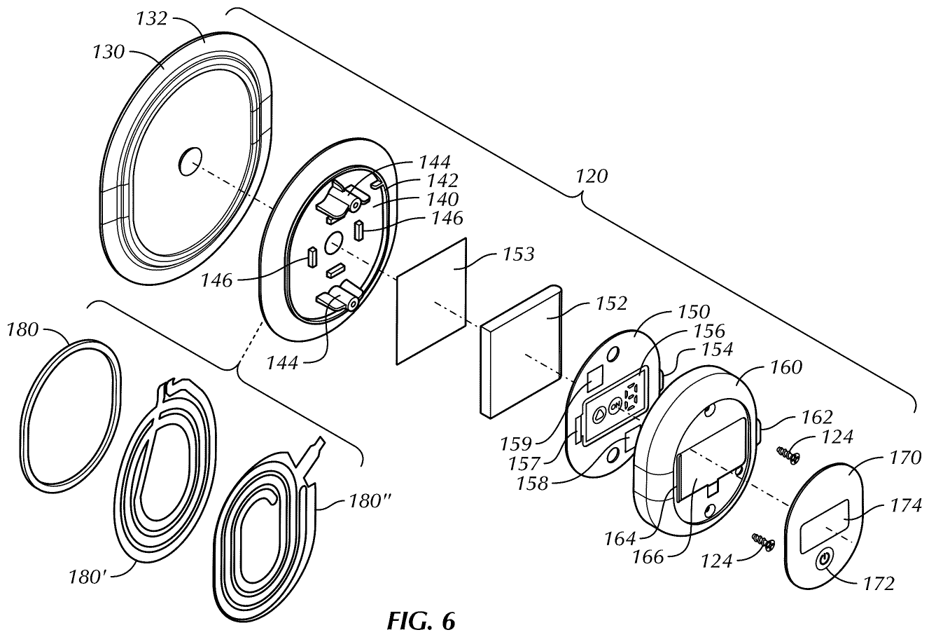

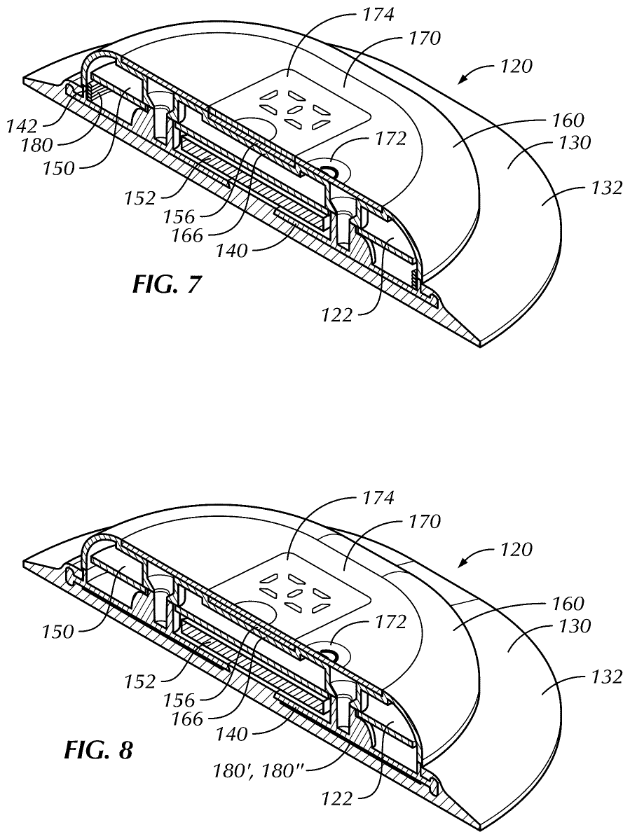

Referring now to FIGS. 6-8, in some examples, the components of the charger 100 include a base 140, a printed circuit board (PCB) 150, and a rechargeable battery 152 to be disposed within an interior 122 of the housing 120. In some examples, the rechargeable battery 152 is disposed within the interior 122 of the housing 120, the battery 152 being configured to store electricity. In some examples, the base 140 and the bottom surface 130 form a bottom side of the housing, the bottom side being configured to face the patient 10 proximate the location of the medical device 50. In some examples, the bottom surface 130 is molded onto the base 140 to form the bottom side of the housing 120. In this way, the bottom surface 130 is securely mounted onto the base 140 to reduce a chance of the bottom surface 130 peeling off of the base 140 during repeated usage and/or increased age of the charger 100. In other examples, the bottom surface 130 can be adhesively coupled to the base 140. In some examples, the bottom surface 130 is formed from a rubber material to provide a comfortable interface between the patient 10 and the charger 100.

The base 140, in some examples, includes one or more stand-offs 146 to provide a mounting location for the rechargeable battery 152 on the base 140. In some examples, an adhesive substrate 153 is used to attach the battery 152 to the one or more standoffs 146. Although the base 140 is shown in FIG. 5 as including four standoffs 146, it should be understood that, in other examples, more or fewer than four standoffs 146 can be used to support the battery 152, depending on the size and shape of the battery, the space constraints within the interior 122 of the housing 120, etc. In some examples, the battery 152 can be directly attached to the base 140 without using the one or more standoffs 146. In some examples, the adhesive substrate 153 includes double-sided tape. In other examples, the adhesive substrate 153 includes an adhesive, such as, but not limited to, epoxy. With the battery 152 in place within the housing 120, in some examples, the battery 152 is disposed just below the PCB 150, and the rechargeable battery 152 is electrically coupled to the PCB 150.

In some examples, the base 140 includes one or more support posts 144 configured to add rigidity to the housing 120 and hold the housing 120 together. Although shown with two support posts 144, it should be understood that more or fewer than two support posts 144 can be used within the housing 120 depending upon size and/or space constraints within the interior 122 of the housing 120 and/or strength requirements of the housing 120. In some examples, the one or more support posts 144 extend from the base 140, through the PCB 150, and to the top surface 160 with one or more fasteners 124 fastening the top surface 160 to the one or more support posts 144 of the base 140. In various examples, the one or more fasteners 124 can include one or more screws, one or more bolts, or the like.

In some examples, with the base 140 attached to the top surface 160, the PCB 150 is disposed between the base 140 and the top surface 160. In some examples, the PCB 150 includes the display 156 coupled to the PCB 150. In some examples, the display 156 is attached to the PCB 150 with a cable 157. In further examples, the display 156 can be fed through a slot 164 in the top surface 160 to place the display 156, while still electrically coupled to the PCB 150 using the cable 157, within a recess 166 in the top surface 160. In this way, with the display 156 within the recess 166 of the top surface, the faceplate 170 can be attached to the top surface 160 such that the window 174 of the faceplate 170 is disposed over the display 156 to allow the display 156 to be seen through the window 174 in the faceplate 170.

In some examples, the charging port 154 is attached to the PCB 150. In some examples, the PCB 150 includes circuitry electrically connecting the charging port 154 with the rechargeable battery 152. In some examples the charging port 154 extends from a side of the PCB 150, such that, with the base 140 attached to the top surface 160 and the PCB 150 disposed between the base 140 and the top surface 160, the charging port 154 extends into the charging feature 162 of the top surface 160. In this way, the charging port 154 is accessible within the charging feature 162 to allow the patient 10 or caregiver to plug a charging cable into the charging port 154 to charge the rechargeable battery 152.

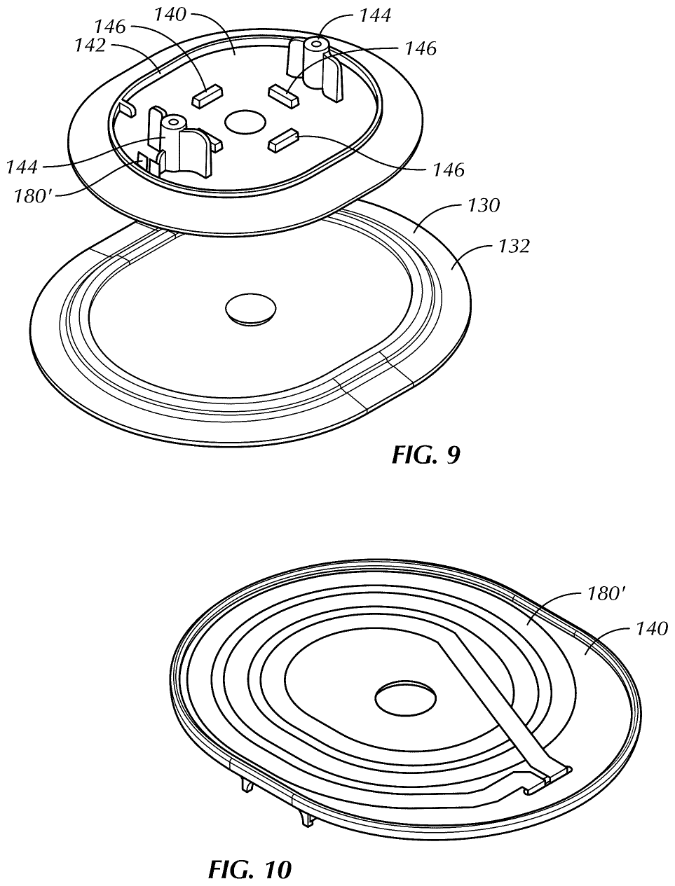

Referring now to FIGS. 1 and 6-10, in some examples, a coil 180, 180′, 180″ is disposed proximate the bottom side (including the bottom surface 130 and the base 140) of the housing 120 and electrically coupled to the battery 152. Although each of the coils 180, 180′, 180″ is shown in FIG. 6, each of the coils 180, 180′, 180″ is not necessarily intended to be disposed within the charger 100 at the same time. That is, in some examples, only one of the coils 180, 180′, 180″ is disposed within the charger 100. However, it is contemplated that, in other examples, the charger 100 can include two or more coils 180, 180′, 180″ at the same time if charging using different coils 180, 180′, 180″ is desirable. In some examples, one of the coils 180, 180′, 180″ is disposed within the charger 100 with the choice of which coil 180, 180′, 180″ is incorporated within the charger 100 being based on one or more factors, such as, but not limited to, case of manufacture, cost of manufacture, implantable medical device 50 to be charged, size and/or shape of the charger 100, etc. Various types of coils 180, 180′, 180″ are contemplated herein. For instance, in some examples, the coil 180 can include a metallic wire wound into a generally elliptical or ovular shape (sized and shaped to fit within the interior 122 of the housing 120). In other examples, the coil 180′ can include a flat metallic ribbon formed into a flattened coil shape sized and shaped to fit within the interior 122 of the housing 120. In still other examples, the coil 180″ can include a flexible printed circuit configured to form a coil and sized and shaped to fit within the interior 122 of the housing 120. The coil 180″, in some examples, connects to a connector on the PCB 150. In some examples, the coil 180″ can be positioned just below the battery 152 in order to reduce the size of the charger 100. The coil 180″ provided in the flexible printed circuit provides a cost effective and repeatable solution that is efficient and compact. In still further examples, the coil can include traces formed in the PCB 150 and configured to function similar to the coils 180, 180′, 180″ but differ in that the coil is integrated within the PCB 150 and is not a separate component from the PCB 150. In some examples, the coil 180, 180′, 180″ is formed from copper. In other examples, the coil 180, 180′, 180″ is formed from other conductive metallic materials instead of or in addition to copper.

In some examples, the coil 180, 180′, 180″ is configured to inductively couple with the implantable medical device 50 with the charger 100 placed against the patient 10 proximate the location of the implantable medical device 50 in order to inductively charge the implantable medical device 50 using electricity from the battery 152. The wireless power, in some examples, is transmitted from the charger 100 to the medical device 50 via inductive coupling with a frequency of 6.78 MHz or 13.56 MHz. In some examples, a control module 158 is disposed within the interior 122 of the housing 120, the control module 158 being configured to selectively supply electricity from the battery 152 to the coil 180, 180′, 180″ in order to inductively charge the implantable medical device 50. In some examples, the control module 158 is included on the PCB 150 with electrical traces, wires, and/or circuitry coupling the control module 158 to the battery 152 and the coil 180, 180′, 180″. In some examples, the control module 158 is controlled by the patient 10 or caregiver to supply electricity to the coil 180, 180′, 180″ to charge the medical device 50, to cease supplying electricity to the coil 180, 180′, 180″ once the medical device 50 has been sufficiently charged, to allow charging of the rechargeable battery 152 within the charger 100 with a charging cable coupled to the charging port 154, or the like.

In some examples, the coil 180, 180′, 180″ is disposed within the interior 122 of the housing 120 proximate the bottom side (including the bottom surface 130 and the base 140) of the housing 120. The closer the coil 180, 180′, 180″ is to the bottom side of the housing 120, the better the inductive coupling of the coil 180, 180′, 180″ to the implantable medical device 50, resulting in more efficient and/or faster charging of the medical device 50 and/or reduced operating temperature of the charger 100 and/or the medical device 50 during charging. As such, in some examples, the coil 180, 180′, 180″ is disposed within the charger 100 as close to the bottom surface of the charger 100 (and as close to the patient 10) as possible.

To that end, in some examples, the coil 180 is disposed proximate and around a side wall 142 of the base 140 of the housing 120 with the coil 180 disposed along the bottom side of the housing 120. In this way, the base 140 and the bottom surface 130 are the only parts of the charger 100 in between the coil 180 and the skin of the patient 10.

In other examples, the coil 180′, 180″ is disposed along the bottom side of the housing 120. For instance, in some examples, the coil 180′, 180″ can be affixed to a bottom of the base 140. Thereafter, the bottom surface 130 can be affixed to the base 140 and the coil 180′, 180″, thereby sandwiching the coil 180′, 180″ between the base 140 and the bottom surface 130. In some examples, the bottom surface 130 is adhered to the base 140 and the coil 180′, 180″. In other examples, bottom surface 130 is molded onto the base 140 and over the coil 180′, 180″, thereby embedding the coil 180′, 180″ within the bottom side, and, more specifically, the bottom surface 130 of the housing 120. In some examples, the coil 180′, 180″ is embedded inside the bottom side of the housing 120 during manufacturing through a two-step injection molding process in which the bottom surface 130 is injection molded over a portion of the base 140, thereby embedding the coil 180′, 180″ within the bottom surface 130 of the housing 120. In other words, the coil 180′, 180″ is molded within a portion of the bottom side of the housing 120. In this way, the bottom surface 130 is the only part of the charger 100 in between the coil 180′, 180″ and the skin of the patient 10, thereby allowing for the coil 180′, 180″ to be placed much closer to the patient 10 than is possible for typical charging devices.

Referring now to FIGS. 5 and 6, in some examples, the charger 100 includes a communication module 159. In some examples, the communication module 159 is included on the PCB 150 with electrical traces, wires, and/or circuitry coupling the communication module 159 to, among other things, the control module 158. In some examples, the communication module 159 is configured to communicate with a mobile device 190, such that, with the mobile device 190 communicatively coupled to the communication module 159, the mobile device 190 is configured to communicate with the control module 158 and control the charger 100. In some examples, the mobile device 190 includes an application or app 192 configured to allow a user (for instance, the patient 10, the caregiver, etc.) to control the charger 100. In various examples, the mobile device 190 can include a smart phone, a smart tablet, a smart watch, a personal digital assistant, a dedicated controller for the charger 100, or the like.

In some examples, the app 192 includes one or more parameter displays 194 and one or more controls 196. In some examples, charger controls and/or wand controls are replaced with the controls of the app 192. In some examples, the one or more parameter displays 194 can include various parameters including, but not limited to, one or more of charge percentage, elapsed charging time, charging time to go, alignment of the charger 100 with the medical device 50, temperature of the charger 100 and/or the medical device 50, a charging history, amount of charge of the rechargeable battery 152 of the charger 100, etc. In some examples, the app 192 allows the user to control and monitor the charging process wirelessly. The user, in some examples, can initiate or stop the charging process from the mobile device 190 without needing to physically interact with the charger 100, which can be particularly useful in situations in which the charger 100 is attached to a hard-to-reach area on the patient 10. Also, in some examples, proper alignment of the charger 100 with the implanted medical device 50 can be verified using the mobile device 190. Also, it is possible for the user to easily check the status of the charge on mobile device 190 and rely on the app 192 for reminders on when to charge the medical device 50.

In some examples, the app 192 can provide the user with the ability to customize one or more monitoring and/or therapy parameters based on the needs of the user. The app 192 can provide real-time battery management features, in some examples, thereby allowing the user to monitor the charging status and battery levels of the medical device 50. In some examples, notifications can be sent to the mobile device 190 of the user, alerting them when the battery 152 is fully charged or when it requires attention, for instance. The mobile device 190, in some examples, can then provide visual indications, sounds, and/or vibration to notify or alert the user of various conditions, parameters, issues, etc. In this way, the user can promptly respond to the charging needs of the medical device 50, thereby maximizing usage and battery life of the medical device 50. In some examples, the app 192 can provide insights into energy consumption patterns, allowing the user to track and monitor energy usage over time, enabling the user to make informed decisions about charging habits and optimize energy efficiency. The app 192, in some examples, can serve as a platform for delivering firmware updates and enhancements to the charger 100 and/or the medical device 50, allowing for continuous improvements, bug fixes, and the addition of new features without requiring physical modifications or replacements.

In addition to communicating with the mobile device 190, the communication module 159, in some examples, is also configured to communicate with the medical device 50 implanted within the patient 10. The mobile device 190, in some examples, can also be configured to communicate directly with the medical device 50 implanted within the patient 10. In some examples, communication between devices (between the mobile device 190 and the charger 100, between the mobile device 190 and the medical device 50, and between the charger 100 and the medical device 50) is done through Bluetooth Low Energy (BLE).

In some examples, the charger 100 reduces, if not eliminates, the need for external charging accessories, providing for greater freedom of movement and convenience for the patient 10 during charging. The patient 10 can wear the charger 100 directly on his or her body, allowing for charging on the go without being tethered to a power source. Traditional chargers include multiple components physically connected through cables, which can become tangled and create a messy charging environment. The charger 100, in some examples, can reduce, if not eliminate, the need for cables, thereby providing a cable-free charging experience. This not only reduces cable clutter but also reduces, if not eliminates, the hassle of untangling cables and minimizes the risk of cable damage.

The present inventors have recognized various advantages of the subject matter described herein. The present inventors have recognized, among other things, that the present inventive subject matter can be used to charge an implantable medical device implanted within a patient while minimally impacting comfort and/or mobility of the patient. In various examples, the present inventive subject matter is advantageous in that it provides a rechargeable charger that allows for cordless charging of the implantable medical device when the charger is sufficiently charged. Moreover, in some examples, the present inventive subject matter provides for a single-component compact charger that can be easily attached to the patient in the location of the implantable medical device and concealed underneath clothing of the patient. In this way, in various examples, the present inventive subject matter is advantageous in that it provides a charger that is relatively easy to attach to the patient, relatively easy to control and monitor, relatively comfortable to wear, and minimally impactful to the mobility of the patient while wearing the charger. While various advantages of the example systems are listed herein, this list is not considered to be complete, as further advantages may become apparent from the description and figures presented herein.

Although the subject matter of the present patent application has been described with reference to various examples, workers skilled in the art will recognize that changes can be made in form and detail without departing from the scope of the subject matter recited in the below claims.

The above Detailed Description includes references to the accompanying drawings, which form a part of the Detailed Description. The drawings show, by way of illustration, specific examples in which the present apparatuses and methods can be practiced. These embodiments are also referred to herein as “examples.”

The above Detailed Description is intended to be illustrative, and not restrictive. For example, the above-described examples (or one or more elements thereof) can be used in combination with each other. Other embodiments can be used, such as by one of ordinary skill in the art upon reviewing the above description. Also, various features or elements can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

In this document, the terms “a” or “an” are used to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “about” and “approximately” or similar are used to refer to an amount that is nearly, almost, or in the vicinity of being equal to a stated amount.

In the appended claims, the terms “including” and “in which” are used as the plain-English equivalents of the respective terms “comprising” and “wherein.” Also, in the following claims, the terms “including” and “comprising” are open-ended, that is, an apparatus or method that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

1. A wireless charger for an implantable medical device, the implantable medical device implantable at a location within a patient, the charger comprising:
a housing including an interior, the housing including a bottom side configured to face the patient proximate the location of the implantable medical device;
a rechargeable battery disposed within the interior of the housing, the battery configured to store electricity; and
a coil disposed proximate the bottom side of the housing and electrically coupled to the battery, the coil configured to inductively couple with the implantable medical device with the charger placed against the patient proximate the location of the implantable medical device in order to inductively charge the implantable medical device using electricity from the battery.

2. The charger of claim 1, comprising a control module disposed within the interior of the housing, the control module configured to selectively supply electricity from the battery to the coil in order to inductively charge the implantable medical device.

3. The charger of claim 2, comprising a communication module configured to communicate with a mobile device, wherein, with the mobile device communicatively coupled to the communication module, the mobile device is configured to communicate with the control module and control the charger.

4. The charger of claim 3, wherein the mobile device includes an application configured to allow a user to control the charger.

5. The charger of claim 1, comprising an adhesive member attached to the charger, the adhesive member configured to adhere the charger to the patient during charging of the implantable medical device.

6. The charger of claim 5, wherein the adhesive member is removably attachable to the charger.

7. The charger of claim 1, wherein the coil is disposed within the interior of the housing proximate the bottom side of the housing.

8. The charger of claim 7, wherein the coil is disposed proximate and around a side wall of the housing.

9. The charger of claim 1, wherein the coil is disposed along the bottom side of the housing.

10. The charger of claim 1, wherein the coil is embedded within the bottom side of the housing.

11. The charger of claim 10, wherein the coil is molded within a portion of the bottom side of the housing.

12. A wireless charger for an implantable medical device, the implantable medical device implantable at a location within a patient, the charger comprising:
a housing including an interior, the housing including a bottom side configured to face the patient proximate the location of the implantable medical device;
an adhesive member attached to the charger, the adhesive member configured to adhere the charger to the patient during charging of the implantable medical device;
a rechargeable battery disposed within the interior of the housing, the battery configured to store electricity;
a control module disposed within the interior of the housing; and
a coil disposed proximate the bottom side of the housing and electrically coupled to the battery, the coil configured to inductively couple with the implantable medical device with the charger placed against the patient proximate the location of the implantable medical device, the control module configured to selectively supply electricity from the battery to the coil in order to inductively charge the implantable medical device.

13. The charger of claim 12, comprising a communication module configured to communicate with a mobile device, wherein, with the mobile device communicatively coupled to the communication module, the mobile device is configured to communicate with the control module and control the charger.

14. The charger of claim 13, wherein the mobile device includes an application configured to allow a user to control the charger.

15. The charger of claim 12, wherein the adhesive member is removably attachable to the charger.

16. The charger of claim 12, wherein the coil is disposed within the interior of the housing proximate the bottom side of the housing.

17. The charger of claim 16, wherein the coil is disposed proximate and around a side wall of the housing.

18. The charger of claim 12, wherein the coil is disposed along the bottom side of the housing.

19. The charger of claim 12, wherein the coil is embedded within the bottom side of the housing.

20. A wireless charger for an implantable medical device, the implantable medical device disposed at a location within a patient, the charger comprising:
a housing including an interior, the housing including a bottom side configured to face the patient proximate the location of the implantable medical device;
an adhesive member removably attachable to the charger, the adhesive member configured to adhere the charger to the patient during charging of the implantable medical device;
a rechargeable battery disposed within the interior of the housing, the battery configured to store electricity;
a control module disposed within the interior of the housing;
a communication module configured to communicate with a mobile device, wherein, with the mobile device communicatively coupled to the communication module, the mobile device is configured to communicate with the control module and control the charger; and
a coil disposed proximate the bottom side of the housing and electrically coupled to the battery, the coil configured to inductively couple with the implantable medical device with the charger placed against the patient proximate the location of the implantable medical device, the control module configured to selectively supply electricity from the battery to the coil in order to inductively charge the implantable medical device.
